SEQUENCE LISTING (17 sequences)

SEQ ID NO: 1 - protein, 22 aa, Artificial Sequence
  Description of Artificial Sequence Synthetic peptide
TPKTRREAEDLQVGQVELGGGP

SEQ ID NO: 2 - protein, 24 aa, Artificial Sequence
  Description of Artificial Sequence Synthetic peptide
EDLQVGQVELGGGPGAGSLQPLAL

SEQ ID NO: 3 - protein, 24 aa, Artificial Sequence
  Description of Artificial Sequence Synthetic peptide
DLQVGQVELGGGPGAGSLQPLALE

SEQ ID NO: 4 - protein, 20 aa, Artificial Sequence
  Description of Artificial Sequence Synthetic peptide
GGGPGAGSLQPLALEGSLQK

SEQ ID NO: 5 - protein, 18 aa, Artificial Sequence
  Description of Artificial Sequence Synthetic peptide
GSLQPLALEGSLQKRGIV

SEQ ID NO: 6 - protein, 17 aa, Artificial Sequence
  Description of Artificial Sequence Synthetic peptide
QPLALEGSLQKRGIVEQ

SEQ ID NO: 7 - protein, 21 aa, Artificial Sequence
  Description of Artificial Sequence Synthetic peptide
ALEGSLQKRGIVEQCCTSICS

SEQ ID NO: 8 - protein, 86 aa, Artificial Sequence
  Description of Artificial Sequence Synthetic proinsulin peptide
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 9 - protein, 11 aa, Artificial Sequence
  Description of Artificial Sequence Synthetic peptide
QPLALEGSLQK

SEQ ID NO: 10 - protein, 26 aa, Artificial Sequence
  Description of Artificial Sequence Consensus peptide
GGGPGAGSLQPLALEGSLQKRGIVEQ

SEQ ID NO: 11 - protein, 28 aa, Artificial Sequence
  Description of Artificial Sequence Synthetic peptide
LAKEWQALCAYQAEPNTCATAQGEGNIK

SEQ ID NO: 12 - protein, 24 aa, Artificial Sequence
  Description of Artificial Sequence Synthetic peptide
KLKVESSPSRSDYINASPIIEHDP

SEQ ID NO: 13 - protein, 20 aa, Artificial Sequence
  Description of Artificial Sequence Synthetic peptide
SFYLKNVQTQETRTLTQFHF

SEQ ID NO: 14 - protein, 20 aa, Artificial Sequence
  Description of Artificial Sequence Synthetic peptide
PKPPKPVSKMRMATPLLMQA

SEQ ID NO: 15 - protein, 21 aa, Artificial Sequence
  Description of Artificial Sequence Synthetic peptide
LLESQIATIEQSAPSQSDQEQ

SEQ ID NO: 16 - protein, 22 aa, Artificial Sequence
  Description of Artificial Sequence Synthetic peptide
AGKSVATNLIGRSLAEKLNSSV

SEQ ID NO: 17 - protein, 23 aa, Artificial Sequence
  Description of Artificial Sequence Synthetic peptide
CQMVSSVDFVPPMAALEEKGILF

(19) United States
(12) Patent Application Publication
(10) Pub. No.: US 2006/0199228 A1
(43) Pub. Date: Sep. 7, 2006

(54) Peptides for treatment of autoimmune diseases

(75) Inventor: Mark Peakman, London (GB)

Correspondence Address: NIXON & VANDERHYE, PC, 901 NORTH GLEBE ROAD, 11TH FLOOR, ARLINGTON, VA, 22203, US

(73) Assignee: King's College London, London (GB)

(21) Appl. No.: 11/370,168

(22) Filed: Mar. 8, 2006

Related U.S. Application Data
(63) Continuation-in-part of application No. 10/783,095, filed on Feb. 23, 2004, now Pat. No. 7,049,292.
(63) Continuation-in-part of application No. PCT/GB05/00236, filed on Jan. 24, 2005.

(30) Foreign Application Priority Data
Jan. 30, 2004 (GB) 0402129.1
Feb. 25, 2004 (GB) 0404199.2

(51) Int. Cl. G01N 33/567; G01N 33/53

(52) U.S. Cl. 435/7.2

(57) ABSTRACT

Peptides for use in the therapy or prevention of Type 1 Diabetes mellitus (T1DM) are those having sequences containing QPLALEGSLQK (SEQ ID NO: 9). Examples are those having a sequence selected from the group consisting essentially of GGGPGAGSLQPLALEGSLQK (SEQ ID NO: 4), GSLQPLALEGSLQKRGIV (SEQ ID NO: 5), and QPLALEGSLQKRGIVEQ (SEQ ID NO: 6). One or more of the above peptides may be combined with one or more peptides having a sequence or sequences consisting essentially of sequences selected from LAKEWQALCAYQAEPNTCATAQGEGNIK (SEQ ID NO: 11), KLKVESSPSRSDYINASPIIEHDP (SEQ ID NO: 12), and SFYLKNVQTQETRTLTQFHF (SEQ ID NO: 13). Also disclosed is a method of assessing the potential of a peptide for T1DM therapy or prevention which comprises subjecting the candidate peptide to a first assay indicative of a pathogenic T cell response in blood or other biological sample, such as an ELISPOT assay for IFN-γ. In the case of a positive response to the first assay, the candidate peptide is subjected to a second assay indicative of a regulatory T cell response to the peptide, such as an ELISPOT assay for IL-10).

This invention relates to autoimmune disease and more particularly to Type 1 diabetes mellitus (T1DM) and latent autoimmune diabetes in adults (LADA). The objective is the treatment of diabetes and other kinds of autoimmune disease using novel peptide combinations and the use of the same peptide combinations in bioassays designed to monitor this, and other diabetes-specific therapies. The invention will be more specifically described in relation to T1DM, but extension of the novel principles described herein for the therapy of other autoimmune diseases will be apparent from the ensuing description.

In T1DM the immune system inadvertently and progressively destroys the cells in the pancreas that make insulin (beta cells). There is thus a loss of immune tolerance to the beta cell. Eventually there are too few beta cells to ensure proper uptake of blood glucose by body cells and the patient has clinical diabetes. The drawbacks of current treatment for this disease are well known, and research effort has been directed over many years to achieving a greater understanding of the disease process with a view to producing improved methods of early diagnosis and more effective therapies. An effective therapy would be one that restores immunological tolerance to the beta cell. This approach would need to be accompanied by a complementary method for the measurement of beta cell tolerance.

It is well understood that the autoimmune attack on beta cells proceeds by way of the MHC class II pathway, in which antigen presenting cells (APCs) process relevant beta cell protein antigens and present their peptide epitopes to CD4+ T lymphocytes, thereby inducing cytokines which assist in the destruction of the beta cells. One approach which has been proposed in the study of T1DM and other autoimmune disease has been to isolate (elute) the effective epitopes from the complex of peptide and HLA class II molecule and to explore the potential of these peptides for diagnosis and therapy. U.S. Pat. No. 5,827,516 is directed to this type of approach for a large number of diseases and U.S. Pat. No. 6,562,943 applies this methodology to T1DM. The literature reference corresponding to U.S. Pat. No. 6,562,516 is Peakman et al, 1999, entitled ‘Naturally processed and presented epitopes of the islet cell autoantigen IA-2 eluted from HLA-DR4’, J. Clin Invest 104:1449-1457. The term IA-2 indicates Islet Tyrosine Phosphatase-like protein.

The entire contents of Peakman et al, identified above, and those of U.S. Pat. Nos. 5,827,516 and 6,562,943 are incorporated herein by reference.

Previous Attempts to Find a Solution

These above mentioned disclosures describe the anticipated utility of these isolated peptides in various ways, for example the following:

(a) As Blocking Peptides

This idea was that a peptide, for example, one which binds to the HLA Class II molecule DR4 (hereinafter HLA-DR4), would bind very strongly to this HLA molecule and displace peptides involved in the disease process, thereby preventing activation of autoreactive T cells. This approach was proposed in 1998, before many of the complexities of antigen processing to derive peptides presented by HLA molecules were fully appreciated. This proposal may therefore be difficult to implement, for the following reasons. The manner in which a natural peptide could block activation of T cells is by competitive exchange so that it occupies all possible HLA binding sites and displaces other occupying peptides. Even the highest binding peptide would struggle to compete to block out all other binders. The peptide would have to compete from outside the cell, where no catalytic enzymes are available to help peptide exchange and where the pH (approximately 7.4 extra—as opposed to 5.0 intra-cellularly) is very unfavourable to peptide exchange. It is estimated that this would require at the very least several milligrams of peptide to get a high enough concentration for effective competition. Since the HLA molecules turn over in a matter of minutes/hours on the cell surface, the competitor peptide would have to be constantly available.

It is highly doubtful whether the system could support this hypothesis and we are aware of no published literature to indicate that such peptides and such a therapeutic application and mode of action have been made and used. Furthermore, such a blocking peptide would be globally immune suppressive, rather than specific for T1DM.

(b) Use of Altered Peptides

What was contemplated in this proposal was to alter the natural peptide sequence so that it would still bind to the HLA molecule but, instead of activating the T cell, would send a slightly different signal, which would either anergise (“switch off”) the T cell, kill the T cell, or switch the T cell to a more benign type. In the 1990s these altered peptide ligands (APL) with antagonistic properties were considered to be highly promising.

The APL approach continues to be pursued in autoimmune disease, although early trials in man have been less encouraging. The problem appears to be that alteration of the natural peptide may render it immunogenic. In two studies in man (in patients with the autoimmune disease multiple sclerosis) the clinical trials were halted because patients developed dangerous allergic immune responses to the APL. These results were reported in the scientific literature in 2000 (refs 1 and 2) below. A further problem was how to identify what alterations would be successful. This proved to be long painstaking work and there was no indication of which peptide to choose, which amino acid to alter and what to change it to.

1. Kappos, L., G. Comi, H. Panitch, J. Oger, J. Antel, P. Conlon, and L. Steinman. 2000. Induction of a non-encephalitogenic type 2 T helper-cell autoimmune response in multiple sclerosis after administration of an altered peptide ligand in a placebo-controlled, randomized phase II trial. The Altered Peptide Ligand in Relapsing MS Study Group. Nat Med 6:1176.

2. Bielekova, B., B. Goodwin, N. Richert, I. Cortese, T. Kondo, G. Afshar, B. Gran, J. Eaton, J. Antel, J. A. Frank, H. F. McFarland, and R. Martin. 2000. Encephalitogenic potential of the myelin basic protein peptide (amino acids 83-99) in multiple sclerosis: results of a phase II clinical trial with an altered peptide ligand. Nat Med 6:1167.

Finally, it is noteworthy that to date no bioassay has been described that is capable of measuring tolerance to beta cells.

General Principles of Invention

In accordance, with the present invention we have avoided approaches of the above kind and have focused research effort on ways of using peptides to suppress the specific cells involved in the development of this disease. We have concentrated on key peptides from preproinsulin. We have developed a multi-step approach, incorporating novel steps and bioassays to show for the first time that certain of these peptide epitopes are crucially involved ir. T1DM/LADA development and may be utilised to achieve natural protection from the disease.

According to a first aspect of the present invention, there is provided a method of selecting a candidate peptide for use in treating or preventing an autoimmune disease, comprising the steps of:

loading antigen presenting cells with an antigen so that peptides derived from the antigen are presented in HLA complexes on the surface of the antigen presenting cells,

purification of said peptides from the HLA complexes;

identification of sequences of the peptides derived from the antigen; and

selection of at least one of the peptides using an assay to determine the recognition of the peptide by pathogenic and/or regulatory CD4+ T lymphocytes.

The HLA complex may include at least one of the following HLA molecules:
HLA-DR2 (DRB1*15), HLA-DR3, HLA-DR4, BLA DQ8, HLA-DQ2. The peptide may be additionally selected on the basis of affinity for a specific HLA molecule, which may be one of the following: HLA-DR2 (DRB1*15), HLA-DR3, HLA-DR4, HLA DQ8, HLA-DQ2. The peptide may be additionally selected on the basis of at least one of (a) beta cell specificity, (b) affinity for HLA-DR4 molecules.

Advantageously the assay is cytokine ELISPOT assay. This assay may detect interferon γ and/or interleukin 10.

The antigen may be selected from preproinsulin, insulinoma associated antigen-2 (IA-2), myelin basic protein, myelin oligodendrocyte glycoprotein, proteolipid protein, collagen, binding immunoglobulin protein, citrullinated filaggrin, glutamic acid decarboxylase-65 (GAD65), Islet-Specific Glucose-6-Phosphatase Catalytic Subunit-Related Protein (IGRP).

In a preferred embodiment, the peptide consists essentially of the sequence of QPALEGSLQK (SEQ ID NO: 9), said sequence being optionally extended by one or more aminoacids bordering said sequence in the consensus sequence GGGPGAGSLQPLALEGSLQKRGIVEQ (SEQ ID NO: 10). For example, the peptide may consist essentially of the sequence GGGPGAGSLQPLALEGSLQK (SEQ ID NO: 4), GSLQPLALEGSLQKRGIV (SEQ ID NO: 5), QPLALEGSLQKRGIVEQ (SEQ ID NO: 6) or GGGPGAGSLQPLALEGSLQKRGIVEQ (SEQ ID NO: 10).

The autoimmune disease may be diabetes. Autoimmune diabetes includes type 1 diabetes mellitus (T1DM) and latent Autoimmune Diabetes in Adults. Alternate terms that have been used for LADA include Late-onset Autoimmune Diabetes of Adulthood, slow onset Type 1 diabetes and Type 1.5 diabetes.

According to a second aspect of the present invention, there is provided a method of treating or preventing an autoimmune disease in patients having at least one HLA encoding allele selected from HLA-DR2 (DRB1*15), HLA-DR4, HLA-DR3, HLA-DQ8, HLA-DQ2 comprising administering to the patient at least one of the candidate peptides identified using the method in any of claims 1 to 12.

The autoimmune disease may be diabetes (T1DM; LADA) and the HLA encoding allele may be HLA-DR4.

The candidate peptide may be administered in combination with at least one peptide selected from LAKEWQALCAYQAEPNTCATAQGEGNIK (SEQ ID NO: 11), KLKVESSPSRSDYINASPIIEHDP (SEQ ID NO: 12), and SFYLKNVQTQETRTLTQFHF (SEQ ID NO: 13).

According to a third aspect of the present invention, there is provided a method of treating or preventing an autoimmune disease in patients having at least one 1LA encoding allele selected from HLA-DR2 (DRB1*15), HLA-DR4, HLA-DR3, HLA-DQ8, HLA-DQ2 comprising extracting antigen presenting cells from a patient;
pulsing the antigen presenting cells with at least one of the candidate peptides identified using the method in any one of claims 1 to 12; and
administering the pulsed antigen presenting cells to the patient.

The autoimmune disease may be diabetes (T1DM; LADA) and the HLA encoding allele may be HLA-DR4.

The antigen presenting cells (APCs) may be additionally pulsed with at least one peptide selected from LAKEWQALCAYQAEPNTCATAQGEGNIK (SEQ ID NO: 11), KLKVESSPSRSDYINASPIWEHDP (SEQ ID NO: 12), and SFYLKNVQTQETRTLTQFHF (SEQ ID NO: 13).

According to a fourth aspect of the present invention, there is provided a method of treating or preventing an autoimmune disease in patients having at least one BLA-DR4 encoding allele, comprising administering to the patient a peptide consisting essentially of the sequence QPLALEGSLQK (SEQ ID NO: 9), said sequence being optionally extended by one or more aminoacids bordering said sequence in the consensus sequence GGGPGAGSLQPLALEGSLQKRGIVEQ (SEQ ID NO: 10).

The peptide may consist essentially of the sequence GGGPGAGSLQPLALEGSLQK (SEQ ID NO: 4), GSLQPLALEGSLQKRGIV (SEQ ID NO: 5), QPLALEGSLQKRGIVEQ (SEQ ID NO: 6). or GGGPGAGSLQPLALEGSLQKRGIVEQ (SEQ ID NO: 10). The method may comprise administering the peptide in combination with at least one peptide selected from LAKEWQALCAYQAEPNTCATAQGEGNIK (SEQ ID NO: 11), KLKVESSPSRSDYINASPIIEHDP (SEQ ID NO: 12), and SFYLKNVQTQETRTLTQFHF (SEQ ID NO: 13). The disease may be diabetes (T1DM; LADA).

According to a fifth aspect of the present invention, there is provided a method of treating or preventing an autoimmune disease in patients having at least one BLA-DR4 encoding allele, comprising
extracting antigen presenting cells from a patient;
pulsing the antigen presenting cells with a peptide consisting essentially of the sequence QPLALEGSLQK (SEQ ID NO: 9), said sequence being optionally extended by one or more aminoacids bordering said sequence in the consensus sequence GGGPGAGSLQPLALEGSLQKRGIVEQ (SEQ ID NO: 10); and
administering the pulsed antigen presenting cells to the patient. The peptide may consist essentially of the sequence GGGPGAGSLQPLALEGSLQK (SEQ ID NO: 4), GSLQPLALEGSLQKRGIV (SEQ ID NO: 5), QPLALEGSLQKRGIVEQ (SEQ ID NO: 6), or GGGPGAGSLQPLALEGSLQKRGIVEQ (SEQ ID NO: 10). The method may comprise additionally pulsing the antigen presenting cells with at least one peptide selected from LAKEWQALCAYQAEPNTCATAQGEGNIK (SEQ ID NO: 11), KLKVESSPSRSDYINASPIIEHDP (SEQ ID NO: 12), and SFYLKNVQTQETRTLTQFHF (SEQ ID NO: 13). The disease may be diabetes (T1DM; LADA).

According to a sixth aspect of the present invention, there is provided a method of monitoring the effectiveness of a therapy administered to patients with, or at risk of an autoimmune disease comprising the steps of:

extracting blood cells from the patient;

incubating the blood cells with at least one peptide selected using the method of any of claims 1 to 12; and

applying a cytokine ELISPOT assay to the incubated cells in order to quantitate the cellular production of cytokines.

The effectiveness of the therapy may be indicated by the presence of an increased number of interleukin 10 producing cells and a reduced number of interferon y producing cells compared to levels present prior to administration of the therapy. The blood cells may be peripheral mononuclear blood cells. The disease may be diabetes (T1DM; LADA).

According to a seventh aspect of the present invention, there is provided a method of assessing the potential of a peptide for use in the therapy or prevention of an autoimmune disease, which comprises subjecting a candidate peptide to a first assay indicative of a pathogenic T cell response in a biological fluid and, where a positive response is obtained, selecting the peptide for optional further assessment.

The first assay may be ELISPOT assay for IFN-γ. In the case of a positive response to the first assay, the candidate peptide may be subjected to a second assay indicative of a regulatory T cell response to the peptide. The second assay may be an ELISPOT assay for IL-10. The autoimmune disease may be diabetes (T1DM; LADA).

The present invention is directed, first, to the problem of how to specifically inactivate the pathogenic CD4+ T lymphocytes responsible for T1DM. This is achieved by (a) identifying the specific peptides recognised by these cells and (b) using them in a therapeutic modality (termed “peptide immunotherapy”). In peptide immunotherapy (PIT) delivery of soluble native peptide leads to the generation/expansion of specialized CD4+ T lymphocytes that are regulatory. Regulatory T cells are capable of specific inhibition of the islet damaging cells by release of anti-inflammatory cytokines, for example interleukin-10 (hereinafter IL-10). Regulatory T cells that operate through release of IL-10 are termed Tr1 cells. Induction of Tr1 cells through PIT is one of the very few therapeutic approaches to offer an outcome in which immunological tolerance to beta cells is restored.

A second problem for which a solution is sought is how to monitor the effect of therapies that are designed to inactivate the CD4+ T lymphocytes responsible for T1DM. Such therapies include PIT, but also other approaches, such as immunosuppressive drugs. This monitoring is achieved by (a) identifying the specific peptides recognised by these CD4+ T lymphocytes and (b) using the peptides in an assay that measures the balance of pathogenic and suppressor CD4+ T lymphocytes through the signature cytokines they make. Such a tolerance assay is critical to the general thrust of preventing T1DM.

Inactivation of pathogenic CD4+ T lymphocytes that recognise specific peptides in the islets is a difficult challenge. Two approaches have been used in the past. The first and most widely used approach attempts to suppress all CD4+ T lymphocytes. Some of these attempts have been successful in showing that therapies aimed at blocking function of CD4+ T lymphocytes can halt progression of diabetes. This is important proof of concept. However, the major problem is that suppressing all CD4+ T lymphocytes leaves the patient open to a very high risk of infection and tumour development as well as the problem of being on the drug long-term with all of the attendant risks that entails. The benefit-to-risk ratio is thus too low for these drugs to be used.

The second approach to inactivating antigen-specific CD4+ T lymphocytes is by administration of the whole antigen, for example by injection or~by nasal spray or orally. This approach is thought to lead to the deletion or suppression of pathogenic T cells. There have been attempts with insulin and the published trials have been unsuccessful.

A third way would be to administer specific peptides from the antigen, either as unaltered peptides or as APLs. Peptides have numerous advantages over the use of whole antigen. Peptides are easy to produce, pharmaceutically formulate and quality assure, they do not carry any of the biological side-effects of the parent molecule and weight for weight provide up to 50 times more of the active component (T cell epitope) than whole antigen. However, there are no studies on beta cell peptides as therapeutics in T1DM in man.

We have solved these problems (i.e which peptides to choose for therapy and how to monitor their beneficial effect) in the following ways.

For the problem of choosing which natural peptides to use for therapy, we have extended the approach described in U.S. Pat. No. 6,562,943 to load APCs with antigen, to allow their internalisation, and to identify the peptides that are naturally processed and presented to CD4+ T lymphocytes. In this approach, we have selected preproinsulin as the putative antigen. We have further extended the approach by the inclusion of an algorithm that involves a series of analytical steps, in which natural peptides are selected on the basis of (a) beta cell specificity; (b) affinity for HLA-DR4; (c) recognition by pathogenic CD4+ T lymphocytes, and (d) recognition by regulatory CD4+ T lymphocytes. This approach yields the identity of those peptides most important in the disease and of the greatest potential for immunotherapy.

The methodology is described below, including reference to accompanying Tables and Figures.

### DESCRIPTION OF FIGURES

FIG. 1. Use of IA-2 and preproinsulin peptides to identify pathogenic (IFN-γ) CD4+ T lymphocytes. Graph shows the percentage of HLA-DR4 cases responding amongst type I diabetes mellitus (T1DM) patients (shaded bars) and control non-diabetic subjects (open bars) to each individual IA-2 and preproinsulin (PI) peptides, as well as the response to combinations of peptides from single or multiple antigens. The greatest discrimination between patients and controls using the least peptides occurs when PI C19-A3 is combined with IA-2 709-36, 752-75 and 853-72 to which 76% of patients and 7% of controls respond (p=0.0001). Patient numbers=25, controls=14.

FIG. 2. Use of IA-2 and preproinsulin peptides and IL-10 ELISPOT to identify non-pathogenic, anti-inflammatory (IL-10 secreting) (protective) CD4+ T lymphocytes. Graph shows the percentage of HLA-DR4 cases responding amongst T1DM patients (shaded bars) and control non-diabetic subjects (open bars) to each individual IA-2 and preproinsulin (PI) peptides, as well as the response to combinations of peptides from single or multiple antigens by production of IL-10 alone. The greatest discrimination between patients and controls using the least peptides occurs when PI C19-A3 is combined with IA-2 709-36, 752-75 and 853-72 to which 64% of patients and 0% of controls respond (p=0.0001). Patient numbers=25, controls=14.

FIG. 3. Use of peptides and IFN-γ and IL-10 ELISPOT to identify pathogenic and protective CD4 T lymphocytes in a single assay format. Development of an assay that discriminates Type 1 diabetes patients from heathy controls on the basis of their polarization of autoreactive CD4+ T lymphocyte responses to IA-2 and PI peptides. Results of cytokine ELISPOT bioassay is shown for patients with T1DM (open circles) and non-diabetic control subjects (closed triangles). For any given positive peptide response (stimulation index ≧3.0 for IFN-γ or IL-10), the stimulation index for each cytokine has been plotted. There is a highly significant inverse correlation between responses represented by each of these cytokines (p=0.000004), indicating extreme polarization of pro-inflammatory and regulatory autoreactivity. Patients with T1DM are clustered close to the y-axis, and non-diabetic control subjects distributed along the x-axis, indicating the association of disease and tolerant states with pro-inflammatory and regulatory responses, respectively.

FIG. 4. The presence of anti-inflammatory (IL-10) CD4+ T lymphocytes delays the onset of diabetes, indicating that these cells have a protective effect through suppression of pathogenic CD4+ T lymphocytes. This is shown by the relationship between age at onset of T1DM and production of IL-10 in response to peptides of IA-2 and preproinsulin. Of patients tested, those making IL-10 responses are significantly older (p=0.01). This provides evidence that anti-inflammatory IL-10 producing CD4+ T lymphocytes that respond to IA-2 and PI, peptides delay diabetes onset.

FIG. 5. Illustration of the sequential multi-step strategy for identification of key preproinsulin peptides for use in peptide immunotherapy.

FIG. 6. Graphs showing the responses to a collection of naturally processed IA-2 and PI peptides. Panel A shows the effect on proinflammatory response (IFNγ secreting cells). Panel B shows the effect on anti inflammatory response (IL-10 secreting cells).

### METHODOLOGY

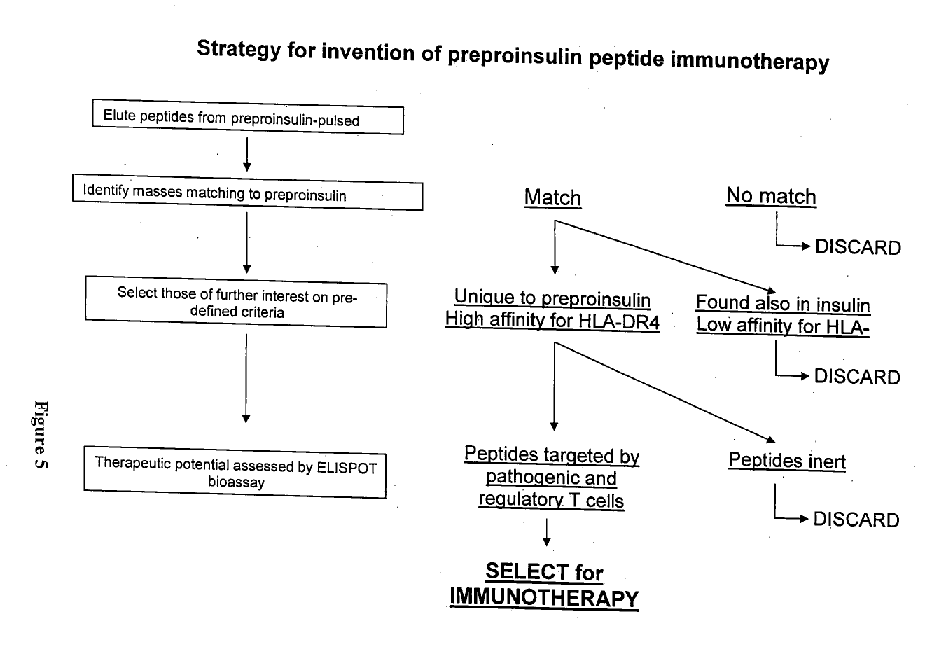

The methodology is detailed according to the strategy/algorithm shown in FIG. 5.

1.1. Identification of Peptides of Preproinsulin Naturally Processed and presented by HLA-DR4

cDNA representing the entire sequence of preproinsulin (embl locus HSPPI, accession X70508.1) was cloned into a pET-12a vector (Novagen Inc, Madison Wis.) modified to include a 6-histidine purification tag and biotinylation sequence at the 5′ end and transformed into BLR(DE3)pLysS competent cells (Novagen Inc) for expression and purification under denaturing conditions followed by refolding using a glutathione redox reaction and confirmation of correct folding by analysis of V8 protease digestion products. Recombinant preproinsulin was delivered to the surface of APCs (Priess Epstein Barr virus (EBV) transformed B cells, homozygous for the Type 1 DM-permissive DRB 1*0401, [DR4/DRw53], DQA1*0301/DQB1*0302 [DQ8] genotype) and HLA-DR4 purified.

Delivery of preproinsulin to the cell surface at high concentration was achieved using an antigen delivery system (ADS), comprising biotinylated pokeweed mitogen (b-PWM) which binds preferentially to carbohydrate moieties on surface receptors with immunoglobulin-like domains, such as the B cell receptor complex on EBV-transformed B cells, as described in Peakman et al, 1999. Avidin was then used as a bridge between cell surface bound b-PWM and biotinylated preproinsulin. Priess EBV B cells were harvested, washed, counted and resuspended at 10⁸/ml on ice. Cells were then pulsed sequentially on ice for 30 minutes with each component of the ADS at optimal concentrations, comprising b-PWM (Sigma Chemical Co, UK; 1 μg/ml), avidin-D (Vector Laboratories; 2 mg/ml) and recombinant biotinylated preproinsulin (20 μg/ml), with two washing steps with excess cold buffer between each pulse. Pulsed Priess cells were then incubated in RPMI 1640/10% FCS (Life Technologies) at 10⁶/ml for 1 or 6 hours at 37° C., 5% CO₂. Under identical conditions, an equivalent number of Priess cells were pulsed with b-PWM/avidin-D but not biotinylated preproinsulin (control cells). At the end of the incubation period, pulsed Priess cells were washed and stored at −80° C.

HLA-DR4 was purified from Priess cell pellets pulsed with the ADS as follows. Briefly, cell pellets were homogenized in hypotonic buffer and a crude membrane fraction solubilized in 4% NP-40 (Sigma Chemical Co.). The detergent-soluble fraction was passed over a series of immunoaffinity columns made with mAbs specific for HLA class I proteins (W6132), HLA-DR (L243) coupled to Protein A-Sepharose and HLA-DQ8 (IVD12) coupled to Affigel-10 (Bio-Rad, Hemel Hempstead, UK). The immunoaffinity columns were eluted with 50 mM glycine, pH 11.5, 0.1% sodium deoxycholate, and immediately neutralized and dialyzed against 10 mM Tris, pH 8.0, 0.1% sodium deoxycholate. HLA-DR4 was >98% pure as assessed by SDS-PAGE. Immediately before acid-extraction of bound peptides, a 0.5 mg aliquot of HLA protein was passed through an HPLC size exclusion column (ProGel TSK G2000 SW, 7.5×300 mm) equilibrated with 10 mM Tris, pH 7.5, to remove molecules not specifically bound to the HLA protein. Fractions containing the HLA-DR4 proteins were concentrated to 100 μl by ultrafiltration (Centricon 10, Amicon, Mass.).

Naturally processed peptide repertoires were acid eluted by incubation for 15 minutes at 70° C. with 800 μl 10% acetic acid and isolated from the remaining HLA protein by ultrafiltration through the Centricon 10. Acid-extracted peptides were vacuum concentrated to approximately 20-30 μl and separated by RP-HPLC, using a microbore C₁₈ column (1.0×250 mm; Vydac, Hesperia, Calif.) at 50-200 μl/minute. Samples were air-dried and approximately 2% loaded onto a sample plate along with 0.4 μl of matrix (α-cyano-4-hydroxycinnamic acid, 10 mg/ml in 50% acetonitrile/0.1% trifluoroacetic acid) and allowed to air dry. Mass spectra were collected at optimum laser intensities by averaging the ion signals from 256 individual scans in both linear and reflector modes using a single stage extended length reflector time-of-flight mass spectrometer (Voyager Elite XL; PerSeptive Biosystems, Framingham, Mass.). Time to mass conversion was performed by external calibration using synthetic peptides. Mass accuracy varied from 0.03% to better than 0.01% in linear and reflector modes, respectively.

The mass spectra for the HLA-DR4 peptide repertoire isolated from Priess cells pulsed with preproinsulin and the control preparation were compared to identify novel m/z values corresponding to peptides derived from preproinsulin. Five masses were identified as being unique to the preproinsulin-pulsed peptide preparation, corresponding to seven preproinsulin sequences (see Table 1). All sequences span an extended region of preproinsulin from the end of the B chain to the middle of the A chain. The peptides circumscribed two potential nested sets that are characteristic of class II MIC processing (C3-C27 and C13-A5).

A second selection step for preproinsulin peptides of therapeutic interest was then applied, using the following strategy. The immune attack in Type 1 diabetes (and LADA) is directed against the β cell alone. Preproinsulin is unique to β cells. In contrast, by virtue of the fact that it is a secreted hormone required for physiological activity throughout the body, insulin (and its discarded, secreted connecting chain, the C-peptide) has a ubiquitous distribution. Eluted peptides were therefore categorised for their presence in preproinsulin alone, versus presence in preproinsulin and insulin/C-peptide. Five of the sequences fulfilled the criterion (SEQ ID NO: 1 and 4-7) of being present in preproinsulin alone.

A further selection step was applied based on the following novel initiative. Only those preproinsulin peptide sequences with high measurable affinity for HLA-DR4 would have peptide immunotherapeutic potential. Synthetic preproinsulin peptides based on the sequences of the five candidates were therefore assessed for their ability to bind soluble HLA-DR4 in vitro in a direct competition binding assay against a biotinylated indicator peptide (98-117 of the MHC class II invariant chain) as follows. HLA-DR4 molecules used in this assay were purified from resting, unmanipulated Priess cell pellets, using the immunoaffinity column approach described above in method 1.1. To perform the binding assay, 12 μg of immunoaffinity-purified HLA-DR4 were incubated with 2.5CM biotinylated indicator peptide and test peptide or non-biotinylated indicator peptide at a range of concentrations (0.1-100 μM) in a final DMSO concentration of 20% in 20 mM 2-[N-morpholino]ethanesulfonic acid, 1% w/v n-octylglucoside, 140 mM sodium chloride, 0.05% sodium azide, pH 5, for 20 hours at room temperature. Peptide mixtures were transferred to wells of a Maxisorp plate (Nalge Nunc, Hereford, UK) that had been pre-coated for 20 hours at room temperature with 100 μl of anti-HLA-DR (L243) capture antibody at 10 μg/ml in phosphate buffered saline (PBS), blocked with 3% non-fat dried milk and 3% bovine serum albumin (BSA) for 30 minutes each and washed 5 times in Tris buffered saline (TBS)/0.1% Tween-20 (all chemicals from Sigma Chemical Company, Poole, Dorset). Plates were incubated for a further 1 hour at room temperature and washed 5 times in TBST. Europium-conjugated streptavidin (Perkin Elmer Ltd., Hounslow, UK) was added at 1 μg/ml in dissociation-enhanced time-resolved fluoroimmunoassays (DELFIA) assay buffer (Wallac Oy, Turku, Finland) and incubated for 45 minutes at room temperature. Wells were washed a further 5 times in TBST and 100 μl DELFIA enhancement solution added to each well. Fluorescent intensity was measured in a DELFIA fluorimeter.

Binding affinity was expressed as an inhibitory concentration 50 (IC₅₀), determined as that required to inhibit binding of 2.5 μM biotinylated indicator peptide by 50%. Only those peptides with high affinity for HLA-DR4 (IC₅₀<10 μM) were selected. One of the nested sets of peptides (C13-A5), containing 3 peptide sequences (SEQ ID NO:4-6) fulfilled these criteria.

The results of this step-wise analysis are shown in Table 1.

Instead of HLA-DR4, the HLA molecule may be HLA-DR2 (DRB1*15), HLA-DR3, HLA DQ8 or HLA-DQ2 for example.

TABLE 1
Experimentally observed and calculated masses of preproinsulin derived peptides
eluted from HLA-DR4, and their matching sequences

| | | | | | IC₅₀ for |
|---|---|---|---|---|---|
| | | Mass | | | binding to |
| Observed | Calculated | accuracy | Residues in | | HLA-DR4 |
| m/z | m/z | (ppm) | preproinsulin | Sequence | (μM) |
| 2336.970 2305.312 2305.312 1836.922 1865.546 1865.546 2224.543 | 2337.216 2305.203 2305.203 1836.981 1866.081 1866.044 2225.072 | 85.8 77.9 77.9 32.3 286.5 267.0 250.0 | B27-C15 C3-C26 C4-C27 C13-C32 C19-A3 C22-A5 C25-A12 | 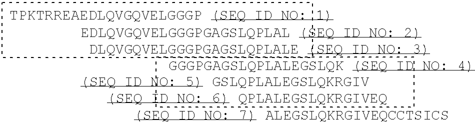 | 50 3 3 5 0.5 0.4 10 |

| | |
|---|---|
| Proinsulin sequence: | |
| 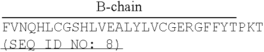 | 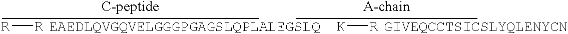 |

Notes: boxes delineate potential nested sets in which the amino acid in bold represents the most likely P1 residue; in the proinsulin sequence, the dibasic motifs R-R and K-R represent the cleavage sites for removal of C-peptide and these residues are subsequently removed by peptidases

The peptides SEQ ID Nos 1 to 7 shown in Table 1 above are those presented by DR4 and derived by cellular processing. It will be understood that these do not constitute epitopes (ie no evidence is provided from elution alone that these peptides are recognised by CD4 T cells) and therefore have no disease relevance taken alone. It cannot be concluded from these data alone that any of these peptides have therapeutic utility for the purposes of the present invention.

1.2 Second Phase of Identification of Peptides of Therapeutic Potential by Analysis of Patient Responses to Candidate Peptides using the IFN-γ ELISPOT.

Our initial screening approach determines which peptides are naturally presented, which have excellent binding characteristics to HLA-DR4, and in the case of preproinsulin, which sequences are unique to this molecule and absent in mature insulin, but not which ones the pathogenic CD4+ T lymphocytes react against during the immune response that leads to T1DM. To solve this problem, we have taken the candidate peptides from preproinsulin and others identified from insulinoma associated antigen-2 (IA-2) disclosed in Peakman et al 1999 and tested each of these individually in an assay format called a cytokine ELISPOT. This detects the signature of a CD4+ T cell according to the cytokine it makes. Making interferon-γ (IFN-γ) represents a pathogenic CD4+ T lymphocyte response. The important peptides from a disease point of view are those that elicit a pathogenic response in this assay. This is therefore a very critical refinement of the simple approach to epitope identification above because it reveals which epitopes are important in the disease context. Prior to the present invention, this approach has not been previously disclosed or carried out with IA-2 peptides or any other peptides.

Interferon-γ Elispot Assay Procedure

Fresh heparinised blood was obtained from 25 Caucasian Type 1 DM patients with HLA-DR4 and acute onset of symptoms, requiring insulin from diagnosis, and from 14 non-diabetic healthy control subjects matched for age and HLA type. Peripheral blood mononuclear cells (PBMCs) were isolated fresh on density gradients (Lymphoprep, Nycom Pharma, Norway) and washed in RPMI 1640 (Life Technologies, Paisley, UK) twice before use. Fresh PBMCs in RPMI 1640 supplemented with antibiotics (TC medium; all Life Technologies) and 10% human AB serum (Harlan SeraLab, Leicestershire, UK) were dispensed into 48-well plates at a density of 2×10⁶ in 0.5 ml supplemented with the selected peptide to a final concentration of 10 μM and incubated at 37° C., 5% CO₂, tilted by 5°. Control wells comprised TC medium containing an equivalent concentration of peptide diluent alone (DMSO), tetanus toxoid (final concentration 10 ng/ml), or PMA/ionomycin (5 ng/ml and 745 ng/ml final concentrations, respectively).

On day +1, 0.5 ml pre-warmed TC medium/10% AB was added and on day +2, non-adherent cells were re-suspended using pre-warmed TC medium/2% AB, washed, brought to a concentration of 1×10⁶/300 μl and 100 μl dispensed in triplicate into wells of 96-well ELISA plates (Nunc Maxisorp, Merck, Poole, UK) pre-blocked with 1% BSA in PBS and pre-coated with monoclonal anti-IFN-γ capture antibody (U-Cytech, Utrecht, NL). After capture at 37° C., 5% CO₂ for 7 hours, cells were lysed in ice cold water, plates washed in PBS/Tween 20 and spots developed using anti-IFN-γ detection antibody and an appropriate revealing agent. Plates were dried and spots of 80-120 μm counted in a BioReader 3000 (BioSys, Karben, Germany). Mean values in test wells were compared with means of the background (DMSO) wells to derive a stimulation index (SI).

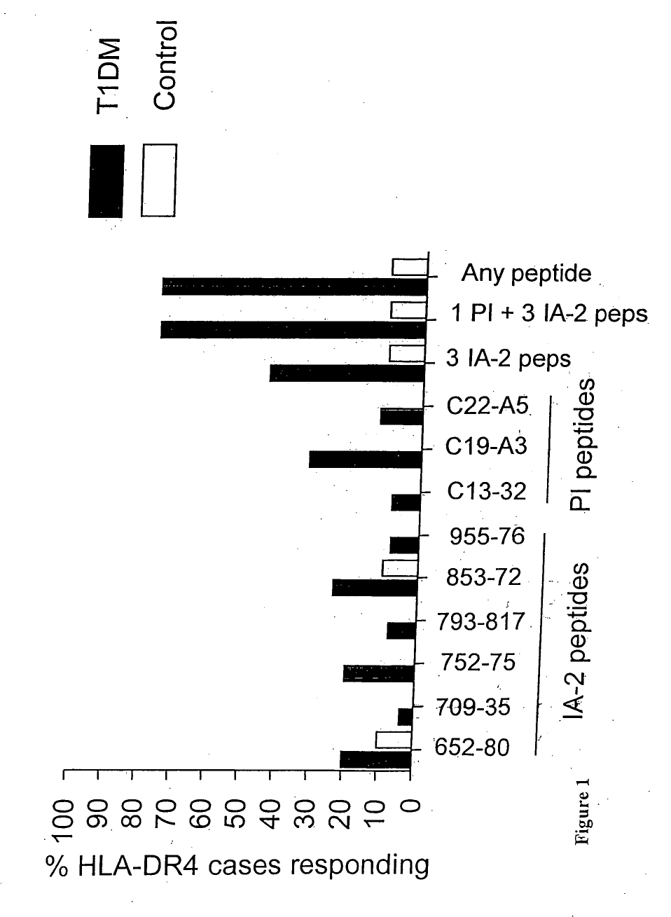

The results are summarised in FIG. 1 and Table 3. FIG. 1 shows the percentage of diabetic patients and controls responding by production of IFN-γ to one or other of the 6 IA-2 peptides and 3 preproinsulin peptides. The results demonstrate that each of the three preproinsulin peptides tested elicits the type of immune response associated with a pathogenic T cell. It is evident that responses are more prevalent in patients and that the greatest discriminative power (between patients and controls) is seen when a minimum of 1 preproinsulin peptide (C19-A3) and 3 IA-2 peptides (709-736, 752-775 and 853-872) are used. In combination, these particular peptides thus represent a cocktail that has the highest achievable disease relevance.

Amongst the 25 patients tested against both IA-2 and PI peptide panels, an IFN-γ response to at least one peptide was seen in 18/25 (72%) T1DM patients, compared with 1/14 (7%) non-diabetic control subjects (p=0.0001). This increase in diagnostic sensitivity was not achieved at the loss of specificity, since none of the non-diabetic control subjects made IFN-γ responses to any of the PI peptides. Overall, responses to the IA-2 and PI peptides, which had been identified by elution from BLA-DR4, tended to be higher in patients with at least one HLA-DR4-encoding allele. Thus, 15/25 (60%) and 10/17 (59%) patients with at least one HLA-DR4 molecule responded to at least one IA-2 or PI peptide respectively, compared with 4/11 (36%) and 4/80 (50%) of patients with non-DR4 alleles, Similarly, the prevalence of responses to either peptide panel was greater amongst those patients with at least one HLA-DR4 allele ( 13/17, 76%) compared with those with no -DR4 alleles (⅝, 63%) although none of these trends were significant with the numbers of cases tested in this study.

Additional studies were carried out using samples from 4 T1DM subjects with islet peptide reactive T cells to examine the nature of the responding cells. Positive responses (SI≧3.0) were entirely abolished when PBMCs were depleted of CD4 T cells, indicating that the autoreactive-T cells detected are CD4+. In addition, we were able to examine the persistence of IFN-γ T cell responses in a further 4 T1DM patients (all DRB1*0401) from whom a second blood sample was available 15-23 weeks after the first. In three patients there was a positive IFN-γ T cell response (SI≧3.0) in the first sample to at least one IA-2 peptide. In two of these patients, the positive responses remained, whilst in the third, the response to one peptide persisted and to the other declined. The fourth patient showed no response in either sample. These results indicate that, when present, pro-inflammatory autoreactive T cell responses have a tendency to persist during the first months after diagnosis.

Summarising these results, there is a clear association between detectable pathogenic IFN-γ responses to selected preproinsulin and IA-2 peptides and the diagnosis of Type 1 diabetes.

TABLE 3
Prevalence of IFN-γ responses to IA-2 and PI peptides in T1DM patients and non-diabetic control subjects

| | | | | | | Responses to Proinsulin peptide | | |
|---|---|---|---|---|---|---|---|---|
| Responses to IA-2 peptide sequences (SI) | | | | | | sequences (SI) | | |
| 652-80 | 709-36 | 752-75 | 793-817 | 853-72 | 955-76 | C13-32 | C19-A3 | C22-A5 |

T1DM patients with HLA-DR4 alleles

| | | | | | | | | | |
|---|---|---|---|---|---|---|---|---|---|
| #1 | | | | | 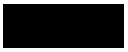 | | | | |
| #2 | 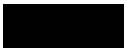 | | | | | | | | |
| #3 | 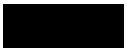 | | 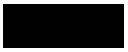 | 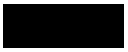 | 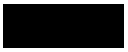 | 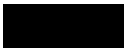 | | 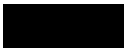 | |
| #4 | | | 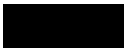 | | 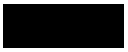 | | | 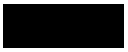 | |
| #5 | 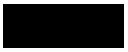 | | | | | | 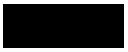 | | 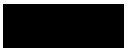 |
| #6 | 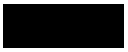 | 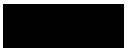 | | | 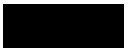 | 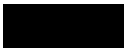 | 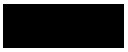 | 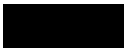 | 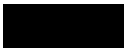 |
| #7 | 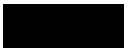 | | | | | | | | |
| #8 | | | | | | | | 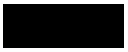 | |
| #9 | | | | | 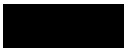 | | | 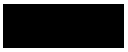 | |
| #10 | | | 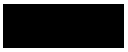 | | | | | 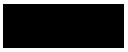 | |
| #11 | | | | | | | | 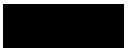 | |
| #12 | | | 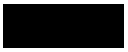 | | | | | 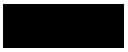 | |
| #13 | | | 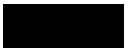 | 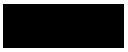 | 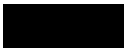 | | | | 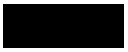 |
| #14 | | | | | | | | | |
| #15 | | | | | | | | | |
| #16 | | | | | | | | | |
| #17 | | | | | | | | | |
| #18 | | | | | | | — | — | — |
| #19 | | 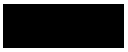 | 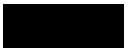 | 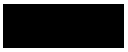 | | 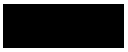 | — | — | — |
| #20 | | | 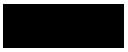 | | | | — | — | — |
| #21 | | | | | | | — | — | — |
| #22 | | | | | | | — | — | — |
| #23 | | | 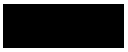 | | | | — | — | — |
| #24 | 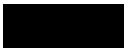 | 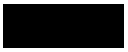 | | | 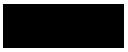 | 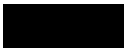 | — | — | — |
| #25 | | | | | | | — | — | — |
| Totals (%) | 6/25 | 3/25 | 8/25 | 3/25 | 7/25 | 4/25 | 2/17 | 8/17 | 3/17 |
| | (24) | (12) | (32) | (12) | (28) | (16) | (12) | (47) | (18) |

T1DM patients with non-DR4 alleles

| | | | | | | | | | |
|---|---|---|---|---|---|---|---|---|---|
| #26 | | | 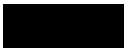 | | | 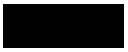 | | 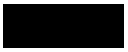 | |
| #27 | 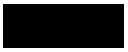 | | | | 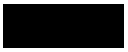 | | | | |
| #28 | | | | | | | 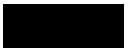 | 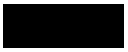 | 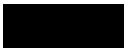 |
| #29 | | | | | | | 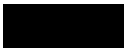 | | 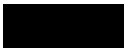 |
| #30 | | | | | | 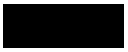 | | 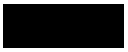 | |
| #31 | | | | | | | | | |
| #32 | | | | | | | | | |
| #33 | | | | | | | | | |
| #34 | | | | | 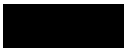 | | — | — | — |
| #35 | | | | | | | — | — | — |
| #36 | | | | | | | — | — | — |
| Totals (%) | 1/11 | 0/11 | 1/11 | 0/11 | 2/11 | 2/11 | 2/8 | 3/8 | 2/8 |
| | (9) | (0) | (9) | (0) | (18) | (18) (25) | (38) | (25) | |

Non-diabetic control subjects

| | | | | | | | | | |
|---|---|---|---|---|---|---|---|---|---|
| C1 | | | | | | | | | |
| C2 | | | | | | | | | |
| C3 | | | | | | | | | |
| C4 | | | | | | | | | |
| C5 | | | | | | | | | |
| C6 | | | | | | | | | |
| C7 | | | | | | | | | |
| C8 | | | | | | | | | |
| C9 | 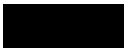 | | | | 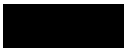 | | | | |
| C10 | | | | | | | | | |
| C11 | | | | | | | | | |
| C12 | | | | | | | | | |
| C13 | | | | | | | | | |
| C14 | | | | | | | | | |
| Totals (%) | 1/14 | 0/14 | 0/14 | 0/14 | 1/14 | 0/14 | 0/14 | 0/14 | 0/14 |
| | (7) | (0) | (0) | (0) | (7) | (0) | (0) | (0) | (0) |

— = not done. SI; stimulation index: see methods for details.
Numbers in shaded boxes indicate SI.

1.3 Third Phase of Identification of Preproinsulin Peptides of Therapeutic Potential by Analysis of Patient Responses to Candidate Peptides using the IL-10 ELISPOT.

In the final step of peptide selection, we sought to identify those peptides with potential efficacy in the manipulation termed “peptide immunotherapy”. As stated previously, this therapeutic approach exerts its effect through the induction/recruitment of specialized regulatory CD4+ T cells (Trl cells) that produce IL-10, and suppress active inflammation in autoimmune disease. Such cells can be detected using the IL-10 ELISPOT. We searched for IL-10 producing, peptide specific Tr1 cells in a group of patients with T1DM, some of whom had early onset of disease and some of whom had late onset of disease.

Our aim was to identify peptides recognised by Tr1 cells in patients with late onset disease. Disease which appears at a later age is likely to reflect slow beta cell destruction, associated with attempts by the immune system to regulate pathogenic T cells (e.g induction of regulatory T cells). We sought to identify peptides recognised by regulatory T cells. The procedure used is identical to that in section 1.2, apart from the following differences:

On day +1, 0.5 ml pre-warmed TC medium/10% AB was added and on day +2, non-adherent cells were re-suspended using pre-warmed TC medium/2% AB, washed, brought to a concentration of 1×10⁶/300 μl and 100 μl dispensed in triplicate into wells of 96-well ELISA plates (Nunc Maxisorp, Merck, Poole, UK) pre-blocked with 1% BSA in PBS and pre-coated with anti-IFN-γ or monoclonal anti-IL-10 capture antibody (U-Cytech, Utrecht, NL). After capture at 37° C., 5% CO₂ for 7 hours, cells were lysed in ice cold water, plates washed in PBS/Tween 20 and spots developed using either anti-IFN-γ or anti-IL-10 detection antibody and an appropriate revealing agent. Plates were dried and spots of 80-120 μm counted in a BioReader 3000 (BioSys, Karben, Germany). Mean values in test wells were compared with means of the background (DMSO) wells to derive a stimulation index (SI).

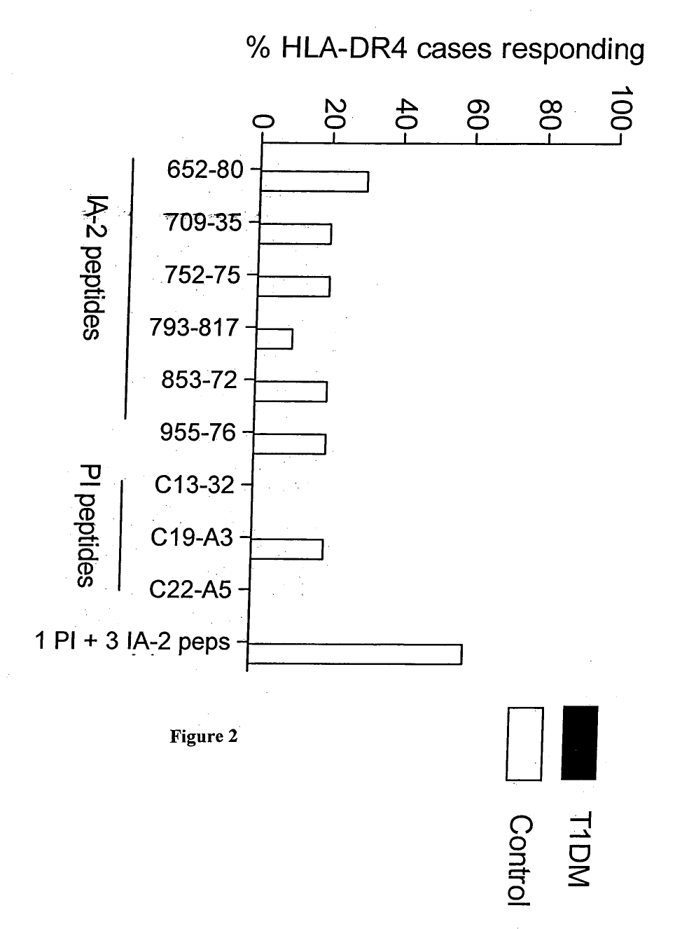

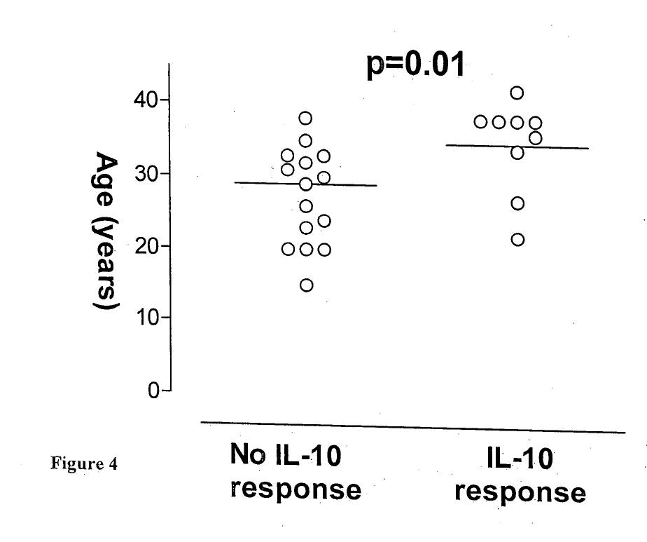

Results are shown in Table 4 and FIGS. 2 and 4. FIG. 2 shows the percentage of diabetic patients and controls responding by production of IL-10 alone to one or other of the 6 IA-2 peptides and 3 preproinsulin peptides. The results demonstrate that each of the three preproinsulin peptides tested elicits the type of immune response associated with a regulatory T cell. It is also evident that responses are more prevalent in non-diabetic patients and that the greatest discriminative power (between patients and controls) is seen when the minimum of 1 preproinsulin peptide (C19-A3) and 3 IA-2 peptides (709-736, 752-775 and 853-872) are used. In combination these peptides thus represent the most relevant to identifying the protective phenotype.

A striking finding was that more than half of the non-diabetic control subjects ( 9/14, 64%) made IL-10 responses to IA-2 peptides, compared with a minority of patients with newly-diagnosed T1DM ( 7/24, 29%; p<0.05, Table 4). These responses were frequently directed against multiple epitopes and of considerable magnitude. Repeated testing one month later in 4 of the non-diabetic control subjects showed that the IL-10 response was reproducible over time (ie 4/4 subjects showed responses classed as positive, SI≧3.0 to the same peptides as in the original assay).

Summarising these data on IL-10 responses, there is a clear trend for an EL-10 response against IA-2 peptides to discriminate patients and control subjects (p<0.05). This trend remains for combined anti-IA-2 and anti-preproinsulin responses (p=0.08) when only the HLA-DR4 cases and controls are considered (consistent with DR4-eluted peptides being more discriminatory amongst DR4 subjects). EL-10 responses to preproinsulin appear non-discriminatory, although fewer cases were studied.

We made the further novel discovery that patients with T1DM who made IL-10 responses to either IA-2 or preproinsulin peptides tended to be significantly older at diagnosis of disease (by an average of 7.5 years) than those who did not (p=0.01; FIG. 4), thus suggesting that this quality of response is associated with a later disease onset, indicating a protective effect of IL-10 production.

In summary, we identified for the first time a series of peptides that are the targets of naturally arising Tr1 cells. The Tr1 cells are clearly associated with two conditions. The first is the healthy, non-diabetic state. The second is the late or slow onset of disease. These results firmly link these Tr1 cells with protection from diabetes development. In this respect, the peptides that are targets of Tr1 cells show ideal properties for use in a peptide immunotherapeutic setting.

In accordance with the present invention, therefore, the use of the novel series of selection steps described above and illustrated in outline in FIG. 5 has enabled us to determine the sequences of crucial peptides and peptide combinations effective for the therapeutic or prophylactic control of T1DM. Three such peptides are those having the following sequences :

| | |
|---|---|
| GGGPGAGSLQPLALEGSLQK, | (SEQ ID NO: 4) |
| GSLQPLALEGSLQKRGIV, | (SEQ ID NO: 5) |
| and | |
| QPLALEGSLQKRGIVEQ | (SEQ ID NO: 6) |

of which that having the sequence GSLQPLALEGSLQKRGIV (SEQ ID NO: 5) is highly preferred. Also comprised within the present invention is a peptide which contains the following consensus of the above three sequences :

GGGPGAGSLQPLALEGSLQKRGIVEQ. (SEQ ID NO: 10)

It is apparent that an essential component sequence of the above peptides is the sequence:

QPLALEGSLQK. (SEQ ID NO: 9)

which sequence is extended at one or both ends thereof in the peptides defined above. The present invention therefore comprises a peptide having a sequence comprising or consisting of QPLALEGSLQK (SEQ ID NO: 9).

As indicated in Table 1, the peptides for use according to the present invention are components of the preproinsulin molecule. No therapeutic activity has previously been ascribed to these novel peptides separated from sequences with which they are associated in preproinsulin. Similarly, no therapeutic activity has been hitherto discovered for peptides having other subsequences of the preproinsulin molecule for which immunogenic properties have been disclosed, including sequences described by Congia et al based solely on experiments in mice ( Proc. Natl. Acad. Sci. U.S.A. vol 95 :3833-3838). In particular, it should be noted that there is no agreement among those skilled in the art as to what peptide components of preproinsulin are immunodominant peptides. In these circumstances, there has been no clear prior indication or suggestion of therapeutic activity associated with peptides having such previously disclosed subsequences. In contrast, the peptides of the present invention have been demonstrated as having therapeutic or preventive activity especially in relation to T1DM. These peptides are especially useful for the treatment of patients that have the HLA-DR4 allele.

Distinction of the inventive peptides from those having the larger sequences as occurring in Nature may be expressed by the term “isolated or purified” peptides (e.g in the senses used in U.S. Pat. No. 6,562,943 B1), although it will be understood that for practical use these peptides will preferably be synthesised and produced to specification in accordance with regulatory requirements. Peptides within the scope of the present invention may also be described in general terms as peptides consisting essentially of at least one of the sequences SEQ ID Nos 4,5,6,9 and 10.

Other possible extensions of the sequence SEQ ID NO :9 are also comprised within the scope of the present invention, along with minor extensions of the peptides SEQ ID Nos 4,5,6,and 10. Extended peptides of this kind which retain the therapeutic potential of the peptides SEQ ID Nos 4,5,6, 9, and 10 may be readily selected by application of selection criteria 1.2 and 1.3 described above.

For use in the therapeutic or prophylactic treatment of T1DM or LADA, at least one of the above peptides will be the primary component of the pharmaceutical composition supplied for such use. Various combinations of two or more of these peptides may be used if desired.

The present invention also comprises one or more of the above sequences in combination with one or more peptides from insulinoma associated antigen-2 (IA-2) that have now been found to exhibit good synergy with preproinsulin peptide C19-A3 (SEQ ID NO 5). These are shown in Table 2 below.

TABLE 2

IA-2 peptides eluted from HLA-DR4 that have
synergy with preproinsulin 75-92

| Numbering in | |
|---|---|
| IA-2 | Sequence |
| 709-36 | LAKEWQALCAYQAEPNTCATAQGEGNIK |
| | (SEQ ID NO: 11) |
| 752-75 | KLKVESSPSRSDYINASPIIEHDP |
| | (SEQ ID NO: 12) |
| 853-72 | SFYLKNVQTQETRTLTQFHF |
| | (SEQ ID NO: 13) |

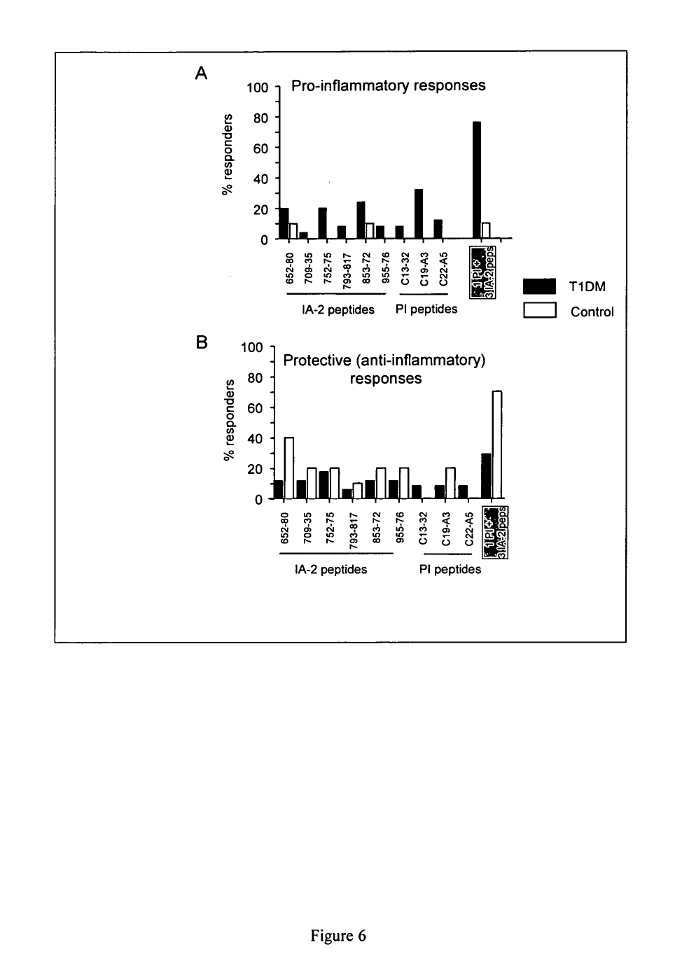

FIG. 6 shows responses to a collection of naturally processed IA-2 and PI peptides. The read-out is for pro-inflammatory cells (interferon-gamma secreting, panel A) and anti-inflammatory (IL-10 secreting, panel B).

It is apparent from the figure that the response to the cocktail of 1 PI and 3 IA-2 peptides is better at identifying pro-inflammatory and anti-inflammatory cells. It follows that any attempt to induce or expand natural T cell regulation using natural peptides will work better, in terms of magnitude of response induced, if more peptide epitopes are used. The combination shown of selected IA-2 and PI peptides is the most powerful in our hands. The peptides used in the cocktail are as follows:

| | | | |
|---|---|---|---|
| IA-2 | 752-775 | (SEQ ID No: 12) | KLKVESSPSRSDYINASPIIEHDP |
| IA-2 | 709-736 | (SEQ ID No: 11) | LAKEWQALCAYQAEPNTCATAQGEGNIK |
| IA-2 | 853-872 | (SEQ ID No: 13) | SFYLKNVQTQETRTLTQFHF |
| Proinsulin | C19-A3 | (SEQ ID No: 5) | GSLQPLALEGSLQKRGIV |

It will be appreciated from the foregoing description that the selection of the above peptides or peptide combinations has resulted from methodology which is also novel and which may be used to determine other possible peptides associated with T1DM or LADA and combinations which will prove effective in the treatment or control of T1DM or LADA. For example, the novel methodology is applicable to the selection of peptides or peptide combinations comprising preproinsulin-derived and IA-2-derived peptides eluted from HLA-DR3, HLA-DQ8 and HLA-DQ2 and Glutamic acid decarboxylase (GAD)65 eluted from HLA-DR4, HLA-DR3, HLA-DQ8 and HLA-DQ2.

Indeed this methodology is of such broad applicability that it permits extension to the search for and selection of therapeutically valuable peptides or peptide combinations derived from autoantigens linked to other autoimmune diseases besides T1DM. Thus, as applied in the T1DM example described in detail herein, the method will begin with elution of peptides held within the corresponding MHC Class II complex and will proceed with selection steps and criteria corresponding to those described herein (1.1, 1.2, and 1.3 above). Examples of such other autoantigens and their diseases to which this methodology is applicable include:

1. In relation to multiple sclerosis, the elution of peptides derived from (i) myelin basic protein; or (ii) myelin oligodendrocyte glycoprotein; or (iii) proteolipid protein from HLA-DR2;

2. In relation to rheumatoid arthritis, the elution of peptides derived from (i) collagen; or (ii) binding immunoglobulin protein (BIP); or (iii) citrullinated filaggrin from HLA-DR4.

Other antigens include glutamic acid decarboxylase-65 (GAD65) and Islet-Specific Glucose-6-Phosphatase Catalytic Subunit-Related Protein (IGRP).

In general terms, therefore, the present invention provides a method of assessing the potential of a peptide for use in the therapy or prevention of an autoimmune disease, which comprises subjecting the candidate peptide to a first assay indicative of a pathogenic T cell response in blood (or other biological sample) (e.g an ELISPOT for IFN-Y) and optionally, in the case of a positive response thereto, subjecting the candidate peptide to a second assay indicative of a regulatory T cell response to the peptide (eg an ELISPOT for IL-10). Where both such assays are used, the peptide giving a positive response in the second assay will be selected for therapy.

In addition to or as an alternative to administering the peptide(s) to the patient, the patient may be treated in the following way.

In the treatment of an autoimmune disease, the peptides identified according to the invention could be employed to induce a regulatory T cell response by using ex vivo pulsing of dendritic cells. Presentation of antigenic peptides by immature dendritic cells are known to favour a regulatory T cell response (Steinman, R. M., Hawiger, D., and Nussenzweig, M. C. 2003. Tolerogenic dendritic cells. Annu Rev Immunol 21:685-711). Dendritic cells from a patient with autoimmune disease could be expanded ex vivo, pulsed with one of the peptides identified according to the invention, and then infused back into the patient. IL-10 ELISPOTs could be used to monitor the appearance of peptide specific regulatory T cells. Likewise, dendritic cells pulsed with the peptide ex vivo could be used to expand regulatory T cells ex vivo, which could then be infused into the patient.

Preparation and Pulsing of Dendritic Cells

The dendritic cells used in this approach would ideally be monocyte-derived, immature myeloid dendritic cells. These can be prepared by a number of standard methods, such as Skowera et al (Skowera, A., de Jong, E. C., Schuitemaker, J. H., Allen, J. S., Wessely, S. C., Griffiths, G., Kapsenberg, M., and Peakman, M. 2005. Analysis of Anthrax and Plague Biowarfare Vaccine Interactions with Human Monocyte-Derived Dendritic Cells. J Immunol 175:7235-7243.). These cells can be maintained and even fixed in an immature state by a variety of chemical means.

Monocyte-derived, immature myeloid dendritic cells would then be pulsed on ice with peptides identified according to the invention at optimal concentrations (1-50 μg/ml), washed and infused into the patient.

Monitoring for the appearance of CD4 regulatory T cells in the peripheral blood secreting IL-10 would carried out as described herein.

2. Tolerance Assay: Development of an Assay to Measure Immunological Tolerance to Beta Cells.

Many/most of the at-risk individuals who may be treated to prevent future diabetes have no symptoms. They are identified as being at risk by a blood test for autoantibodies and genes, either as part of a population-wide screening programme, or because they have a close relative with diabetes. If they have no symptoms or signs, one cannot know whether the therapy is having an effect, without having to wait 5-10 years to see whether they develop diabetes or not. In other words, the whole field of intervention therapy in diabetes needs surrogate markers of therapeutic efficacy (“tolerance assays”). Having identified appropriate peptides for use as described above, we have now developed a bioassay that measures tolerance; the balance of pathogenic and suppressor CD4+ T lymphocytes. No such assay has been available hitherto.

The procedures used are those described in sections 1.2 and 1.3 above, to perform a cytokine ELISPOT on peripheral blood for the combined detection of IFN-γ and HW-10.

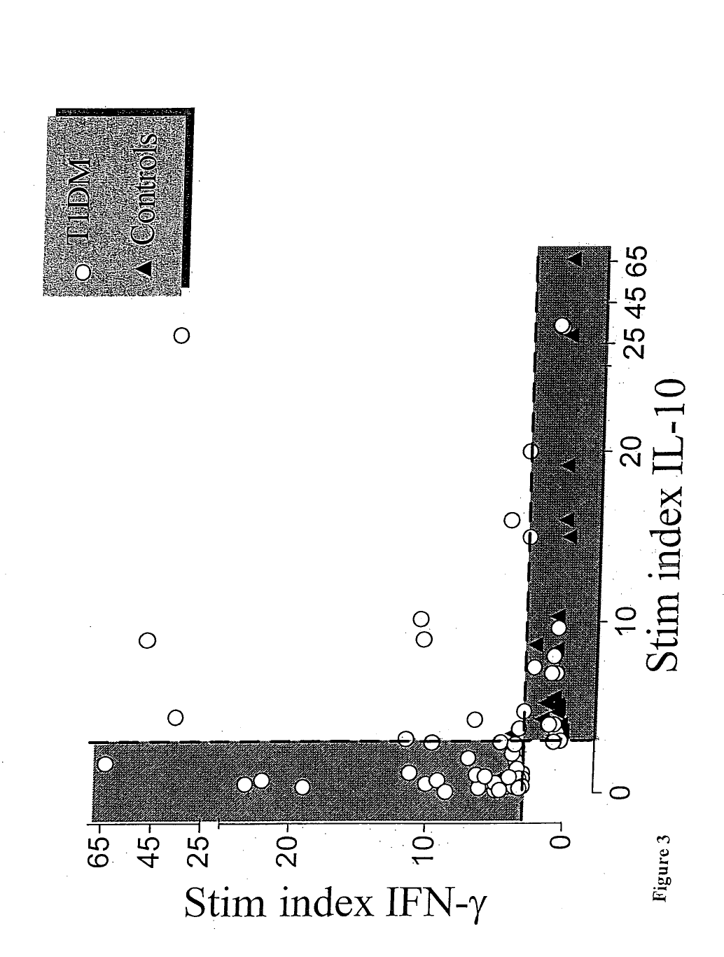

The results of the combined assay analysis on fresh heparinised blood obtained from 25 Caucasian Type 1 DM patients with HLA-DR4 and acute onset of symptoms, requiring insulin from diagnosis, and from 14 non-diabetic healthy control subjects matched for age and HLA type are plotted in FIG. 3. To examine the nature of the relationship between IL-10 and IFN-γ responses to IA-2 and preproinsulin peptides in patients and control subjects, we plotted the stimulation index for each cytokine when a positive peptide response was observed (SI>3.0 for IFN-γ or IL-10). These results demonstrated a highly significant inverse correlation between responses represented by each of these cytokines (FIG. 3; p=0.000004), indicating that in the context of an autoreactive T cell response there is extreme polarization of pro-inflammatory versus regulatory autoreactivity. Moreover, whilst patients with T1DM were clustered close to the y-axis, non-diabetic control subjects were distributed along the x-axis, highlighting the association of the disease and tolerant states with pro-inflammatory and anti-inflammatory or regulatory responses, respectively. In contrast, there was no inverse correlation between IFN-γ and EL-10 responses to tetanus toxoid (p=0.64).

This tendency to make either polarized Thl or regulatory T cell responses to naturally processed and presented IA-2 and preproinsulin epitopes provides a clear distinction in the quality of autoreactivity between T1DM patients and non-diabetic subjects (p<0.0001).

Thus the tolerance assay we describe, when plotted as in FIG. 3, indicates that the combination of the selected peptides and an assay that measures EFN-γ and IL-10 responses can discriminate patients and control subjects into 3 broad categories. Category 1, along the x-axis (L-10 but no IFN-γ), is the healthy non-diabetic state. Category 2, along the y-axis (IFN-γ but no IL-10), is the disease state. Category 3, in the upper right quadrant (IFN-γ and IL-10) is the slowly progressive disease state.

This novel approach identifies responses in categories 1 and 3 as representing measurable degrees of tolerance. Patients with new-onset or pre-diabetes, undergoing immunotherapy and in whom tolerance is being measured using this assay, will be predicted to make a shift from category 2, to the right and down (reader is referred to FIG. 3), as an indication of tolerance induction and treatment effect.

TABLE 4
Prevalence of IL-10 responses to IA-2 and PI peptides in T1DM patients and non-diabetic control
subjects

| | | | | | | Responses to preproinsulin | | |
|---|---|---|---|---|---|---|---|---|
| Responses to IA-2 peptide sequences (SI) | | | | | | peptide sequences (SI) | | |
| 652-80 | 709-36 | 752-75 | 793-817 | 853-72 | 955-76 | C13-32 | C19-A3 | C22-A5 |

T1DM patients with HLA-DR4 alleles

| | | | | | | | | | |
|---|---|---|---|---|---|---|---|---|---|
| #2 | | | | | | | | | |
| #3 | 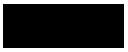 | | 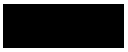 | 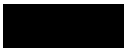 | | 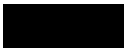 | 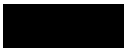 | 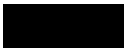 | 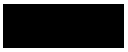 |
| #4 | | | | | | | | | |
| #5 | | 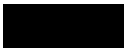 | | | | | | | |
| #6 | | | | | 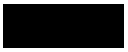 | | | | |
| #7 | | | | | | | | | |
| #11 | | | | | | | | | |
| #12 | | | 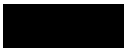 | | | | | | |
| #13 | | | | | | | | | |
| #15 | | | | | | | | | |
| #18 | | | | | | | | | |
| #19 | | | | | | | | | |
| #20 | | | | | | | — | — | — |
| #21 | | | | | | | — | — | — |
| #23 | | | | | | | — | — | — |
| #24 | 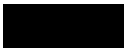 | 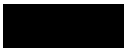 | 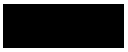 | | 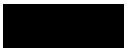 | 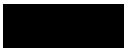 | — | — | — |
| #25 | | | | | | | — | — | — |
| Totals | 2/17 | 2/17 | 3/17 | 1/17 | 2/17 | 2/17 | 1/12 | 1/12 | 1/12 |
| (%) | (12) | (12) | (18) | (6) | (18) | (6) | (8) | (8) | (8) |

T1DM patients with non-DR4 alleles

| | | | | | | | | | |
|---|---|---|---|---|---|---|---|---|---|
| #26 | | | | | | | | | |
| #27 | | 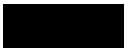 | 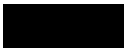 | 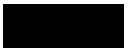 | 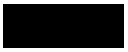 | 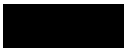 | | 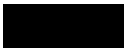 | 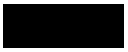 |
| #28 | | | | | | | 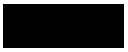 | | |
| #29 | | | | | | | | | |
| #33 | | | | | | | | 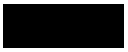 | |
| #35 | 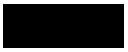 | | 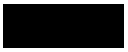 | | 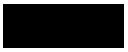 | | — | — | — |
| #36 | | | | | | | — | — | — |
| Totals | 1/7 | 1/7 | 2/7 | 1/7 | 2/7 | 1/7 | 1/5 | 2/5 | 1/5 |
| (%) | (14) | (14) | (29) | (14) | (29) | (14) | (20) | (40) | (20) |

Non-diabetic control subjects

| | | | | | | | | | |
|---|---|---|---|---|---|---|---|---|---|
| C1 | | | | | | | | | |
| C2 | | | | | | | | | |
| C3 | | | 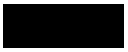 | | | | | | |
| C4 | 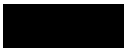 | 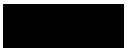 | | 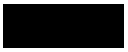 | | 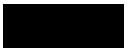 | — | — | — |
| C5 | | | | | 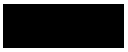 | | | 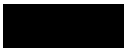 | |
| C6 | | 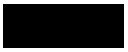 | | | | | | | |
| C7 | | | | | | | | | |
| C8 | 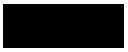 | | 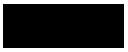 | | 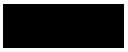 | 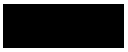 | | 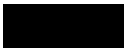 | |
| C9 | 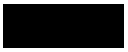 | | | | | | | | |
| C10 | 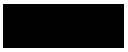 | | | | | | | | |
| C11 | | | | | | 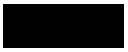 | | | |
| C12 | | | | | | | | | |
| C13 | | 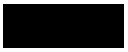 | | 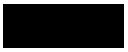 | | | 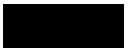 | 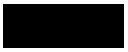 | 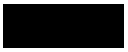 |
| C14 | | | | | | | | | |
| Totals | 4/14 | 3/14 | 2/14 | 2/14 | 2/14 | 3/14 | 1/13 | 3/13 | 1/13 |
| (%) | (29) | (21) | (14) | (14) | (14) | (21) | (8) | (23) | (8) |

— = not done. SI; stimulation index: see methods for details; Numbers in shaded boxes indicate SI.

(1) Specific Therapy for Type 1 Diabetes.

We have made a discovery that provides strong support for the pursuit of this novel strategy. When analysing responses of PBMCs from patients with Type I diabetes to the preproinsulin and IA-2 peptides, we identified these peptides as the targets of a regulatory T cell subset. These Tr1 cells were found in patients with slowly progressive disease. This provides in vivo evidence that Tr1 cells recognising the preproinsulin and IA-2 peptides have regulatory and tolerogenic properties. Since Tr1 cells recognizing these peptides may be induced by peptide immunotherapy, we propose the therapeutic use of preproinsulin and IA-2 peptides in diabetes prevention.

Our strategy for peptide immunotherapy is the use of peptide injection, or related methods, to induce in vivo regulatory populations of T cells (Tr1 cells) that recognise the same peptide as the equivalent effector pathogenic T cells (these cells make IFN-γ and are called Th1). Peptide immunotherapy is particularly potent at inducing Tr1 cells that synthesise the immunosuppressive chemical mediator IL-10. Under these circumstances, when one of the preproinsulin and IA-2 peptides is presented by an antigen presenting cell in the pancreas or local lymph nodes in a patient developing diabetes, the peptide is recognised simultaneously by Th1 and Tr1 cells. It is known that under these circumstances the Tr1 cell is dominant and exercises “bystander suppression” over the Th1 response.

The identification of a specific combination of peptides that identifies pathogenic CD4+ T lymphocytes in a majority of patients leads us to a therapy by which CD4+ T lymphocytes involved in T1DM are inactivated, restoring long-term beta cell tolerance. An important element in this approach is the demonstration that the combination of 4 epitopes from 2 autoantigens is outstanding in terms of its coverage of pathogenic CD4+ T lymphocyte responses. Such coverage gives a greater potency to the therapy, and applicability to a wider range of patients, than any mono-therapy hitherto proposed. Thus our therapeutic approach is multi-epitope, multi-antigen peptide immunotherapy and uses the peptides we have identified through a combination of elution and bioassay.

An example of an aspect of the invention is as follows. The selected peptides are synthesized to GMP grade and pooled in order to represent the best possible combined efficacies. Peptides are used singly or pooled in vials containing up to about 1 mg of each peptide per single dose e.g from 0.5 to 5 to 50 to 250 or up to 500 μg in sterile saline and the vial contents administered. In general, and as used in this example, administration can be by parenteral or oral or topical routes including intradermal, subcutaneous or intravenous injection, or nasally or orally or epicutaneously as simple solutions. Peptides may also be given in conjunction with tolerance-promoting adjuvants or tolerance promoting cells. Tolerance promoting adjuvants include IL-10 and recombinant cholera toxin B-subunit (rCTB), which are co-administered with peptide. Tolerance promoting cells include immature dendritic cells and dendritic cells treated with vitamin D3, (1alpha,25-dihydroxyvitamin D3) or its analogues. In this example, immature dendritic cells are expanded from patient blood in vitro using standard techniques before the commencement of therapy. Peptides are then bound to the dendritic cells in vitro before administration, which may be by any of the parenteral routes mentioned above. In this example, the administration of peptide in any of these forms takes place on 3 occasions at times 0, 1 and 2 months.

In this example, treatment may be continued according to the indication of primary outcome measures. The primary outcome measures are a change in peptide-induced IL-10+ (increase) and IFN-γ+ (decrease) peptide-reactive cells detected by the cytokine ELISPOT assay or similar changes in IL-10+ and IFN-γ+ cells reactive with epitopes of preproinsulin, IA-2 that had not been administered (ie so-called bystander effects). Further primary outcome measures will be changes in basal and stimulated C-peptide levels at 3, 6, and 12 months after commencing treatment and changes in insulin dosage and HbA1c versus placebo, each of which represent enhancement of endogenous insulin production. Any such favourable outcome measures will dictate cessation of therapy; conversely, continuation of presence of or reappearance of, for example IFN-γ+ cells recognising the therapeutic peptides, will dictate continuation of therapy.

In this example, subjects for the therapy are individuals identified as being at-risk of diabetes development in the next 5-10 years through the presence of circulating autoantibodies. Autoantibodies used for this identification are those against preproinsulin, IA-2 and GAD65 and also an autoantibody termed islet cell antibody (ICA). All subjects will have at least one high risk HLA molecule, for example HLA-DR4, -DR3, -DR2 (DRB1*15) -DQ8, -DQ2. Subjects can also be newly-diagnosed subjects with Type 1 diabetes, within 3 months of diagnosis and at least one circulating autoantibody as specified above.

(2) A Tolerance Assay to Monitor Therapy for Type 1 Diabetes.

Also, in accordance with a further aspect of the present invention, we describe hereinafter a tolerance assay that is made up of our peptides plus a cytokine ELISPOT bioassay for use in the monitoring of intervention therapies in patients with, or at risk of Type 1 diabetes. Our identification of specific peptides and combinations of peptides has led to the solution of previous therapeutic problems, in that the peptides can be used (a) to reveal the presence of pathogenic CD4+ T lymphocytes in patients and (b) to reveal the presence of non-pathogenic suppressor CD4+ T lymphocytes that have been induced by preventive therapies. An important contribution is our demonstration that our highly preferred combination of 4 epitopes from 2 autoantigens is vastly superior in terms of its coverage of pathogenic and suppressive CD4+ T lymphocyte responses than any other peptides previously proposed. Thus our diagnostic approach is multi-epitope, multi-antigen screening to monitor the balance of pathogenic versus protective immune responses in patients undergoing therapeutic interventions for Type 1 diabetes.

An example of this approach is as follows. Peptides representing the epitopes having the sequences identified hereinbefore are synthesized by standard Fmoc chemistry to LMP grade and used singly or pooled into cocktails representing the best possible combined efficacies. In this example, a particular immune modulating treatment is commenced with the aim of halting or preventing the autoimmune processes that lead to Type 1 diabetes. An example of this intervention is a course of treatment with peptide immunotherapy, or the non-depleting monoclonal anti-CD3 antibody hOKT3 directed against T cells or an immune suppressive drug such as rapamycin. These therapies are administered for a defined period and then surrogate markers are measured in a tolerance assay to assess the effect of the therapy on pathogenic autoimmunity. An example of a surrogate marker to be used in this way is the cytokine ELISPOT detecting pathogenic (IFN-γ) and suppressor (IL-10) CD4+ T lymphocyte responses to single or cocktails of peptides identified as described above. Reduction or disappearance of pathogenic CD4+ T lymphocytes, or induction of suppressor CD4+ T lymphocytes would lead to a reduction or cessation of therapy. No change or a worsening of these surrogate markers would lead to continuation of therapy and/or the introduction of new reagents.

A further aspect of the invention therefore comprises a method of measuring the state of immunological tolerance of a patient to beta cells or cells involved in other autoimmune disease which comprises the following steps:

(a) Extracting the patient's peripheral blood mononuclear cells

(b) Culturing these cells with any of the peptides or peptide combinations defined hereinbefore, or derived by the methodology described hereinbefore

(c) Applying a cytokine ELISPOT analysis to the cultured cells in order to quantitate the cellular production of cytokines eg interferon-γ and interleukin-10. The patients immunological tolerance to the cells is demonstrated by the presence of an increased number of interleukin-10 producing cells and a reduced number of interferon-γ producing cells.

1. A method of selecting a candidate peptide for use in treating or preventing an autoimmune disease, comprising the steps of:
loading antigen presenting cells with an antigen so that peptides derived from the antigen are presented in HLA complexes on the surface of the antigen presenting cells, purification of said peptides from the HLA complexes;
identification of sequences of the peptides derived from the antigen; and
selection of at least one of the peptides using an assay to determine the recognition of the peptide by pathogenic and/or regulatory CD4+ T lymphocytes.

2. A method as claimed in claim 1, wherein the HLA complexes include at least one of the following HLA molecules:
HLA-DR2 (DRB1*15), HLA-DR3, HLA-DR4, HLA DQ8, HLA-DQ2.

3. A method as claimed in claim 1, wherein the peptide is additionally selected on the basis of affinity for a specific HLA molecule.

4. A method as claimed in claim 3, wherein the HLA molecule is selected from one of the following: HLA-DR2 (DRB1 *15), HLA-DR3, HLA-DR4, HLA DQ8, HLA-DQ2.

5. A method as claimed in claim 1, wherein the assay is a cytokine ELISPOT assay.

6. A method as claimed in claim 5, wherein the assay detects interferon γ.

7. A method as claimed in claim 5, wherein the assay detects Interleukin 10.

8. A method as claimed in claim 1, wherein the antigen is selected from preproinsulin, insulinoma associated antigen-2 (IA-2), myelin basic protein, myelin oligodendrocyte glycoprotein, proteolipid protein, collagen, binding immunoglobulin protein, citrullinated filaggrin, glutamic acid decarboxylase-65 (GAD65), Islet-Specific Glucose-6-Phosphatase Catalytic Subunit-Related Protein (IGRP).

9. A method as claimed in claim 1, wherein the peptide is additionally selected on the basis of at least one of (a) beta cell specificity, (b) affinity for HLA-DR4 molecules.

10. A method as claimed in claim 1, wherein the selected peptide consists essentially of the sequence of QPALEGSLQK (SEQ ID NO: 9), said sequence being optionally extended by one or more aminoacids bordering said sequence in the consensus sequence GGGPGAGSLQPLALEGSLQKRGIVEQ (SEQ ID NO: 10).

11. A method as claimed in claim 1, wherein the selected peptide consists essentially of the sequence GGGPGAGSLQPLALEGSLQK (SEQ ID NO: 4), GSLQPLALEGSLQKRGIV (SEQ ID NO: 5), QPLALEGSLQKRGIVEQ (SEQ ID NO: 6), or GGGPGAGSLQPLALEGSLQKRGIVEQ (SEQ ID NO: 10).

12. A method as claimed in claim 1 wherein the autoimmune disease is diabetes (type 1 diabetes mellitus (Ti DM); latent autoimmune diabetes in adults (LADA).

13. A method of treating or preventing an autoimmune disease in patients having at least one HLA encoding allele selected from HLA-DR2 (DRB1*15), HLA-DR4, HLA-DR3, HLA-DQ8, HLA-DQ2 comprising administering to the patient at least one of the candidate peptides identified using the method in claim 1.

14. A method as claimed in claim 13, wherein the autoimmune disease is diabetes (Ti DM; LADA), and wherein the HLA encoding allele is HLA-DR4.

15. A method as claimed in claim 13, comprising administering the candidate peptide in combination with at least one peptide selected from LAKEWQALCAYQAEPNTCATAQGEGNIK (SEQ ID NO: 11), KLKVESSPSRSDYINASPIIEHDP (SEQ ID NO: 12), and SFYLKNVQTQETRTLTQFHF (SEQ ID NO: 13).

16. A method of treating or preventing an autoimmune disease in patients having at least one HLA encoding allele selected from HLA-DR2 (DRB1*15), HLA-DR4, HLA-DR3, HLA-DQ8, HLA-DQ2 comprising extracting antigen presenting cells from a patient;
pulsing the antigen presenting cells with at least one of the candidate peptides identified using the method in claim 1; and
administering the pulsed antigen presenting cells to the patient.

17. A method as claimed in claim 16 wherein the autoimmune disease is diabetes (T1DM; LADA), and wherein the HLA encoding allele is HLA-DR4.

18. A method as claimed in claim 16 comprising additionally pulsing the antigen presenting cells with at least one peptide selected from LAKEWQALCAYQAEPNTCATAQGEGNIK (SEQ ID NO: 11), KLKVESSPSRSDYINASPIIEHDP (SEQ ID NO: 12), and SFYLKNVQTQETRTLTQFHF (SEQ ID NO: 13).

19. A method of treating or preventing an autoimmune disease in patients having at least one HLA-DR4 encoding allele, comprising administering to the patient a peptide consisting essentially of the sequence QPLALEGSLQK (SEQ ID NO: 9) said sequence being optionally extended by one or more aminoacids bordering said sequence in the consensus sequence GGGPGAGSLQPLALEGSLQKRGIVEQ (SEQ ID NO: 10).

20. A method as claimed in claim 19, wherein the peptide consists essentially of the sequence GGGPGAGSLQPLALEGSLQK (SEQ ID NO: 4), GSLQPLALEGSLQKRGIV (SEQ ID NO: 5), QPLALEGSLQKRGIVEQ (SEQ ID NO: 6), or GGGPGAGSLQPLALEGSLQKRGIVEQ (SEQ ID NO: 10).

21. A method as claimed in claim 19 comprising administering the peptide in combination with at least one peptide selected from LAKEWQALCAYQAEPNTCATAQGEGNIK (SEQ ID NO: 11), KLKVESSPSRSDYINASPIIEHDP (SEQ ID NO: 12), and SFYLKNVQTQETRTLTQFHF (SEQ ID NO: 13).

22. A method as claimed in claim 19, wherein the autoimmune disease is diabetes (T1DM; LADA).

23. A method of treating or preventing an autoimmune disease in patients having at least one HLA-DR4 encoding allele, comprising
extracting antigen presenting cells from a patient;
pulsing the antigen presenting cells with a peptide consisting essentially of the sequence QPLALEGSLQK (SEQ ID NO: 9), said sequence being optionally extended by one or more aminoacids bordering said sequence in the consensus sequence GGGPGAGSLQPLALEGSLQKRGIVEQ (SEQ ID NO: 10); and
administering the pulsed antigen presenting cells to the patient.

24. A method as claimed in claim 23 wherein-the peptide consists essentially of the sequence GGGPGAGSLQPLALEGSLQK (SEQ ID NO: 4), GSLQPLALEGSLQKRGIV (SEQ ID NO: 5), QPLALEGSLQKRGIVEQ (SEQ ID NO: 6), or GGGPGAGSLQPLALEGSLQKRGIVEQ (SEQ ID NO: 10).

25. A method as claimed in claim 23 comprising additionally pulsing the antigen presenting cells with at least one peptide selected from LAKEWQALCAYQAEPNTCATAQGEGNIK (SEQ ID NO: 11), KLKVESSPSRSDYINASPIIEHDP (SEQ ID NO: 12), and SFYLKNVQTQETRTLTQFHF (SEQ ID NO: 13).

26. A method as claimed in claim 23 wherein the autoimmune disease is diabetes (T1DM; LADA).

27. A method of monitoring the effectiveness of a therapy administered to patients with, or at risk of an autoimmune disease comprising the steps of:
extracting blood cells from the patient;
incubating the blood cells with at least one peptide selected using the method of claim 1; and
applying a cytokine ELISPOT assay to the incubated cells in order to quantitate the cellular production of cytokines.

28. A method as claimed in claim 27, wherein the effectiveness of the therapy is indicated by the presence of an increased number of interleukin 10 producing cells and a reduced number of interferon y producing cells compared to levels present prior to administration of the therapy.

29. A method as claimed in claim 27 wherein the blood cells are peripheral mononuclear blood cells.

30. A method as claimed in claim 27 wherein the autoimmune disease is diabetes (T1DM; LADA).

31. A method of assessing the potential of a peptide for use in the therapy or prevention of an autoimmune disease, which comprises subjecting a candidate peptide to a first assay indicative of a pathogenic T cell response in a biological fluid and, where a positive response is obtained, selecting the peptide for optional further assessment.

32. A method according to claim 31, in which the first assay is an ELISPOT assay for IFN-γ.

33. A method according to claim 31, in which, in the case of a positive response to the first assay, the candidate peptide is subjected to a second assay indicative of a regulatory T cell response to the peptide.

34. A method according to clam 32, in which the second assay is an ELISPOT assay for IL-10.

35. A method according to claim 31, in which the autoimmune disease is diabetes (T1DM; LADA).
